(19) United States
(12) Patent Application Publication
(10) Pub. No.: US 2021/0147367 A1
(43) Pub. Date: May 20, 2021

(54) CRYSTALLINE FORMS OF A MAGL INHIBITOR

(71) Applicants: Lundbeck La Jolla Research Center, Inc., San Diego, CA (US); H. Lundbeck A/S, Valby (DK)

(72) Inventors: Cheryl A. GRICE, Encinitas, CA (US); Daniel J. BUZARD, San Diego, CA (US); Michael B. SHAGHAFI, San Diego, CA (US); Nicole S. WHITE, San Diego, CA (US); Heidi LOPEZ DE DIEGO, Nærum (DK); Frans Dennis THERKELSEN, Holbæk (DK); Ida Marie Brødsgaard KNUDSEN, Valby (DK)

(21) Appl. No.: 17/096,721

(22) Filed: Nov. 12, 2020

Related U.S. Application Data
(60) Provisional application No. 62/936,126, filed on Nov. 15, 2019.

(51) Int. Cl. C07D 241/04

(52) U.S. Cl. CPC C07D 241/04; C07B 2200/13

(57) ABSTRACT

Described herein is new crystalline forms of the MAGL inhibitor 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof.

### CROSS-REFERENCE

This application claims benefit of U.S. Provisional Application No. 62/936,126, filed on Nov. 15, 2019, which is herein incorporated by reference in its entirety.

### FIELD OF THE INVENTION

The present invention relates to crystalline forms of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof which is a monoacylglycerol lipase (MAGL) inhibitor for use in the treatment of various diseases and disorders which is believed to be linked to the regulation of endocannabinoid system signalling activities. Furthermore, the present invention relates to 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment of various diseases and disorders which is believed to be linked to the regulation of endocannabinoid system signaling activities.

### BACKGROUND OF THE INVENTION

Monoacylglycerol lipase (MAGL) is a member of the serine hydrolase superfamily. MAGL is expressed throughout the brain, in neurons, microglia, astrocytes, and oligodendrocytes. MAGL is the primary enzyme controlling the degradation of 2-arachidonoylglycerol (2-AG) to arachidonic acid (AA) (Blankman et al. Chem Biol. 2007; Nomura et al. Science. 2011).

2-AG is the most abundant endocannabinoid ligand in the brain where it acts as a retrograde messenger to reduce excessive neurotransmission via the activation of pre-synaptic CB₁ receptors (Kano et al. Physiol Rev. 2009; Katona and Freund. Physiol Rev. 2009), regulating immune response via the activation of microglial CB₂ receptors (Turcotte et al. Cell Mol Life Sci. 2016), and promote neuroprotection via e.g., its effects on oligodendrocyte production and survival (Ilyasov et al. Front Neurosci. 2018).

AA is one of the most abundant fatty acids in the brain and the main precursor of eicosanoids such as prostanoids and leukotrienes that are known inflammatory mediators.

MAGL is at the crossroads between the endocannabinoid and eicosanoid signalling systems. Inhibiting the action or activation of MAGL is a promising therapeutic approach for the prevention or treatment of brain disorders whose pathological hallmarks include excessive neurotransmission, neuroinflammation or neurodegeneration such as Alzheimer's disease (AD), Parkinson's disease (PD), Huntington's disease (HD), multiple sclerosis (MS), amyotrophic lateral sclerosis (ALS), traumatic brain injury, stroke, epilepsy, pain, migraine, addiction, anxiety, depression and other stress-related disorders (Grabner et al. Pharmacol Ther. 2017; Mulvihill et al. Life Sci. 2013; Gil-Ordõñez et al. Biochem Pharmacol. 2018). To move a drug candidate to a viable pharmaceutical product it can be important to understand whether the drug candidate has polymorphic forms as well as the thermodynamic stability of these forms to choose candidates suitable for further development.

### SUMMARY OF THE INVENTION

Accordingly in a first aspect of the invention is provided a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof.

In a further aspect of the invention is provided a pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, and one or more pharmaceutically acceptable carriers or diluents.

In a further aspect of the invention is provided a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment of a disease or a disorder selected from pain, epilepsy/seizure disorder, Alzheimer's disease, Huntington's chorea, Huntington's disease, multiple sclerosis, obsessive-compulsive disorder, Parkinson's disease, depression, Post-traumatic stress disorder, generalized anxiety disorder, persistent motor tic disorder, persistent vocal tic disorder, and dystonia.

In a further aspect of the invention is provided a method for the treatment of a disease or disorder such as pain, epilepsy/seizure disorder, Alzheimer's disease, Huntington's chorea, Huntington's disease, multiple sclerosis, obsessive-compulsive disorder, Parkinson's disease, depression, Post-traumatic stress disorder, generalized anxiety disorder, persistent motor tic disorder, persistent vocal tic disorder, and dystonia; which method comprises the administration of a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof.

In a further aspect of the invention is provided a use of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof in the manufacture of a medicament for the treatment of a disease or disorder selected from pain, epilepsy/seizure disorder, Alzheimer's disease, Huntington's chorea, Huntington's disease, multiple sclerosis, obsessive-compulsive disorder, Parkinson's disease, depression, Post-traumatic stress disorder, generalized anxiety disorder, persistent motor tic disorder, persistent vocal tic disorder, and dystonia.

Other objects, features and advantages of the compounds, methods, and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### INCORPORATION BY REFERENCE

All publications and patent applications mentioned in this specification are herein incorporated by reference to the extent applicable and relevant.

### BRIEF DESCRIPTION OF THE FIGURES

FIG. 1 Illustrates an X-ray powder diffraction (XRPD) pattern of Compound (I) form A. X-axis: ° 2θ; Y-axis: intensity (counts).

FIG. 2 Illustrates a thermo-gravimetric analysis (TGA) thermogram of Compound (I) form A. X-axis: Temperature (° C.); Y-axis: Weight (%).

FIG. 3 Illustrates a Differential scanning calorimetry (DSC) thermogram of Compound (I) form A: X-axis: Temperature (° C.); Heat flow (Normalized) (W/g).

FIG. 4 Illustrates an X-ray powder diffraction (XRPD) pattern of Compound (I) form C. X-axis: ° 2 θ Y-axis: intensity (counts).

FIG. 5 Illustrates a thermo-gravimetric analysis (TGA) thermogram of Compound (I) form C. X-axis: Temperature (° C.); Y-axis: Weight (%).

FIG. 6 Illustrates a Differential scanning calorimetry (DSC) thermogram of Compound (I) form C: X-axis: Temperature (° C.); Heat flow (Normalized) (W/g).

FIG. 7 Illustrates an X-ray powder diffraction (XRPD) pattern of Compound (I) form D. X-axis: ° 2 θ; Y-axis: intensity (counts).

FIG. 8 Illustrates a thermo-gravimetric analysis (TGA) thermogram of Compound (I) form D. X-axis: Temperature (° C.); Y-axis: Weight (%)

FIG. 9 Illustrates a Differential scanning calorimetry (DSC) thermogram of Compound (I) form D: X-axis: Temperature (° C.); Heat flow (Normalized) (W/g).

FIG. 10 Illustrates an X-ray powder diffraction (XRPD) pattern of Compound (I) form E. X-axis: ° 2 θ; Y-axis: intensity (counts).

FIG. 11 Illustrates a thermo-gravimetric analysis (TGA) thermogram of Compound (I) form E. X-axis: Temperature (° C.); Y-axis: Weight (%).

FIG. 12 Illustrates a Differential scanning calorimetry (DSC) thermogram of Compound (I) form E: X-axis: Temperature (° C.); Heat flow (Normalized) (W/g).

FIG. 13 Illustrates a Differential scanning calorimetry (DSC) thermogram of Compound (I) form E: X-axis: Temperature (° C.); Heat flow (Normalized) (W/g) within the temperature range of 25° C. to 130° C. (prior to major peak).

FIG. 14 Illustrates an X-ray powder diffraction (XRPD) pattern of Compound (I) hydrate. X-axis: ° 2θ; Y-axis: intensity (counts).

FIG. 15 Illustrates a thermo-gravimetric analysis (TGA) thermogram of Compound (I) hydrate. X-axis: Temperature (° C.); Y-axis: Weight (%).

FIG. 16 Illustrates a dynamic vapor sorption (DVS) sorption kinetic plot of Compound (I) hydrate. X-axis: Time (min); Y-axis (left): Change in mass (%); Y-axis (right): Relative humidity (%).

FIG. 17 Illustrates a Differential scanning calorimetry (DSC) thermogram of Compound (I) hydrate: X-axis: Temperature (° C.); Heat flow (Normalized) (W/g).

FIG. 18 Illustrates an X-ray powder diffraction (XRPD) pattern of Compound (I) Na.H₂O. X-axis: ° 2θ; Y-axis: intensity (counts).

FIG. 19 Illustrates a thermo-gravimetric analysis (TGA) thermogram of Compound (I) Na.H₂O. X-axis: Temperature (° C.); Y-axis: Weight (%)

FIG. 20 Illustrates a differential scanning calorimetry (DSC) thermogram of Compound (I) Na.H₂O. X-axis: Temperature (° C.); Y-axis: normalized Heat flow (W/g).

FIG. 21 Polarization Microscopy of Compound (I) form A.

FIG. 22 Polarization Microscopy of Compound (I) Na.H₂O.

### DETAILED DESCRIPTION OF THE INVENTION

The compound, 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, designated herein as Compound (I), has the structure:

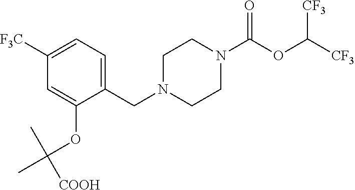 [SMILES: CC(C)(Oc1cc(C(F)(F)F)ccc1CN1CCN(C(=O)OC(C(F)(F)F)C(F)(F)F)CC1)C(=O)O]

Compound (I) is a MAGL inhibitor exhibiting a IC₅₀ equal to or less than 50 nM. The experimental protocol can be found in example 14.

The present invention relates to crystalline forms of Compound (I), and the use of the compounds for treating various disease and disorder which is believed to be linked to the regulation of endocannabinoid system signaling activities. The present invention further provides crystalline forms of Compound (I) described herein as “Compound (I) form A”, “Compound (I) form C”, “Compound (I) form D”, “Compound (I) form E”, and “Compound (I) hydrate”. These are free forms of Compound (I). The term “free form” refers to Compound (I) in non-salt form. The term “hydrate” refers to substance containing Compound (I), but also containing molecules of water incorporated into the crystal lattice. An additional crystalline form of Compound (I) is further described herein, wherein Compound (I) is in the form of a crystalline pharmaceutically acceptable salt. The term pharmaceutically acceptable salts include salts formed with inorganic and/or organic bases, such as alkali metal bases, such as sodium hydroxide, lithium hydroxide, potassium hydroxide, alkaline earth bases, such as calcium hydroxide and magnesium hydroxide, amino acids such as Lysine and Arginine and organic bases, such as trimethylamine, triethylamine.

Further described herein is a sodium salt monohydrate form of Compound (I) designated as “Compound (I) Na.H₂O”.

In an embodiment of the invention Compound (I) Na.H2O has the following structure:

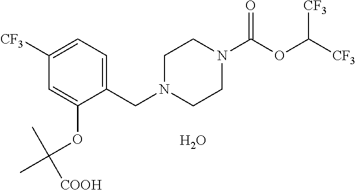

The term “pharmaceutically acceptable salts” in reference to Compound (I) refers to a salt of Compound (I), which does not cause significant irritation to a mammal to which it is administered and does not substantially abrogate the biological activity and properties of the compound.

It should be understood that a reference to crystalline forms of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt includes the solvent addition forms (solvates) unless stated specifically otherwise. Solvates contain either stoichiometric or non-stoichiometric amounts of a solvent, and are formed during the process of product formation or isolation with pharmaceutically acceptable solvents such as water (e.g. hydrates), ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. Hydrates can be formed when the solvent is water or by absorption of water from the surroundings.

While not intending to be bound by any particular theory, certain solid forms have different physical and chemical properties, e.g., stability, solubility and dissolution rate, appropriate for pharmaceutical and therapeutic dosage forms. Moreover, while not wishing to be bound by any particular theory, certain solid forms have different physical and chemical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy and thermal analysis), as described herein and known in the art. DSC analysis showed Compound (I) form A to be the most stable free form up to 60° C., while Compound (I) form D is more thermodynamically stable at higher temperatures.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, “amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of Compound (I), age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “inhibits”, “inhibiting”, or “inhibitor” of an enzyme as used herein, refer to inhibition of enzymatic activity.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “prophylactically effective amount,” as used herein, refers that amount of a composition applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “subject” as used herein, refers to an animal which is the object of treatment, observation or experiment. By way of example only, a subject may be, but is not limited to, a mammal including, but not limited to, a human. In an embodiment, the subject is a human.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC₅₀ refers to an amount, concentration or dosage of a particular test compound that achieves a 50% inhibition of a maximal response, such as inhibition of MAGL, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

### Embodiments of the Invention

In the following, embodiments of the invention are disclosed. The first embodiment is denoted E1, the second embodiment is denoted E2 and so forth.

E1. A crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof.

E2. The crystalline form according to embodiment 1, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form A having at least one of the following properties:

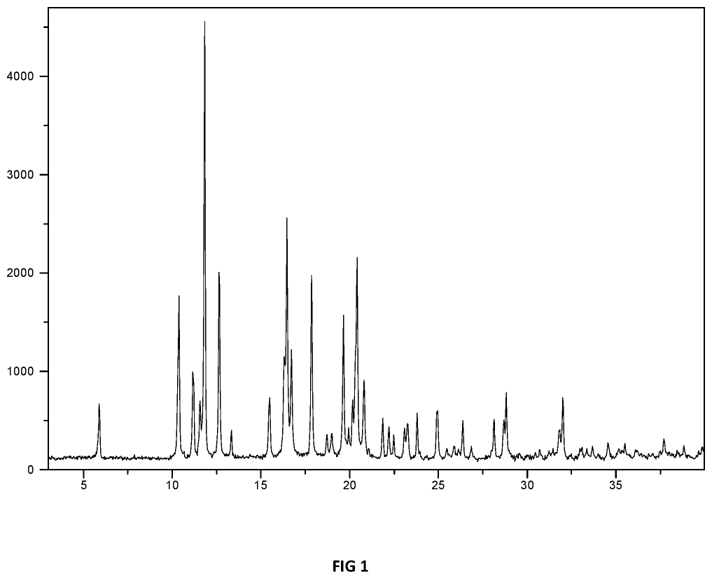

a) an X-ray powder diffraction (XRPD) obtained using CuK_{α1} radiation (λ=1.5406 Å) showing an XRPD pattern substantially the same as shown in FIG. 1;

b) an X-ray powder diffraction (XRPD) pattern obtained using CuK_{α1} radiation (λ=1.5406 Å) showing characteristic peaks at the following 2θ-angles: 10.36, 16.45, 16.71, 19.66, 21.85, and 24.93°;

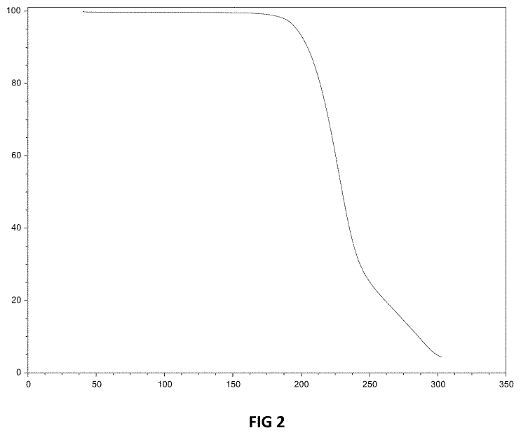

c) a thermo-gravimetric analysis (TGA) substantially similar to the one set forth in FIG. 2; or

d) combination thereof.

E3. The crystalline form according to embodiment E2, wherein said crystalline form has a crystal form characterized by an XRPD obtained using CuK_{α1} radiation (λ=1.5406 Å) showing peaks at the following 2θ-angles: 10.36, 16.45, 16.71, 19.66, 21.85, and 24.93°.

E4. The crystalline form according to embodiment E2, wherein said crystalline form has a crystal form characterized by an XRPD obtained using CuK_{α1} radiation (λ=1.5406 Å) showing an XRPD pattern substantially the same as shown in FIG. 1.

E5. The crystalline form of embodiment E2, wherein the crystalline form has a thermo-gravimetric analysis (TGA) substantially similar to the one set forth in FIG. 2.

E6. The crystalline form according to embodiment E1, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form C having at least one of the following properties:

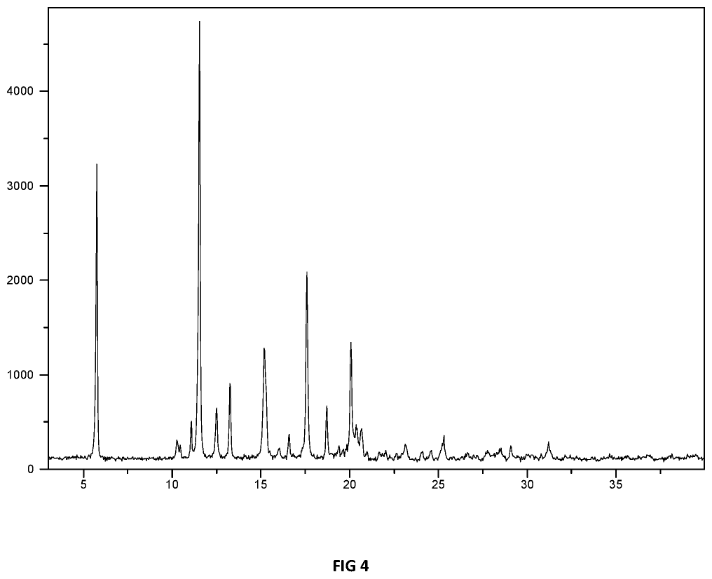

a) an X-ray powder diffraction (XRPD) obtained using CuK_{α1} radiation (λ=1.5406 Å) showing an XRPD pattern substantially the same as shown in FIG. 4;

b) an X-ray powder diffraction (XRPD) pattern obtained using CuK_{α1} radiation (λ=1.5406 Å) showing characteristic peaks at the following 2θ-angles: 5.75, 11.08, 12.50, 15.21, 17.58, and 20.06°;

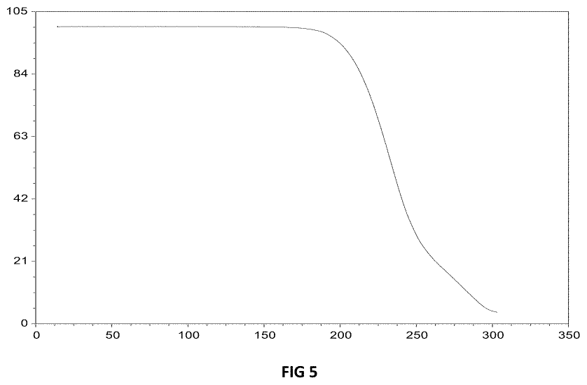

c) a thermo-gravimetric analysis (TGA) substantially similar to the one set forth in FIG. 5; or

d) combination thereof.

E7. The crystalline form according to embodiment E6, wherein said crystalline form has a crystal form characterized by an XRPD obtained using CuK_{α1} radiation (λ=1.5406 Å) showing peaks at the following 2θ-angles: 5.75, 11.08, 12.50, 15.21, 17.58, and 20.06°.

E8. The crystalline form according to embodiment E6, wherein said crystalline form has a crystal form characterized by an XRPD obtained using CuK_{α1} radiation (λ=1.5406 Å) showing an XRPD pattern substantially the same as shown in FIG. 4.

E9. The crystalline form according to embodiment E6, wherein the crystalline form has a thermo-gravimetric analysis (TGA) substantially similar to the one set forth in FIG. 5.

E10. The crystalline form according to embodiment E1, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form D having at least one of the following properties:

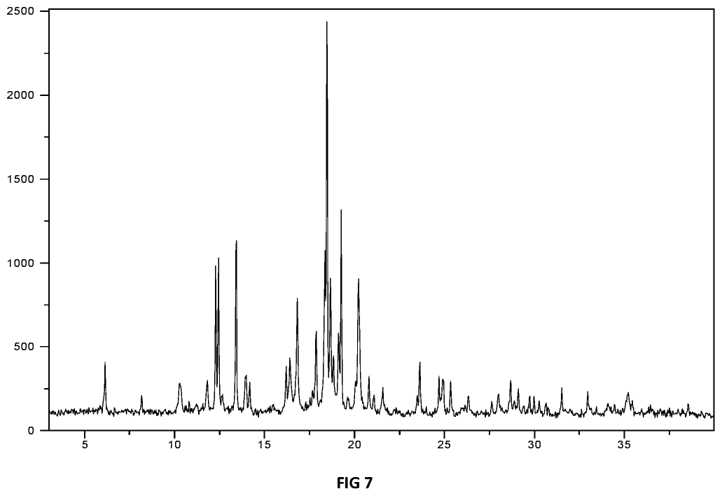

a) an X-ray powder diffraction (XRPD) obtained using CuK_{α1} radiation (λ=1.5406 Å) showing an XRPD pattern substantially the same as shown in FIG. 7;

b) an X-ray powder diffraction (XRPD) pattern obtained using CuK_{α1} radiation (λ=1.5406 Å) showing characteristic peaks at the following 2θ-angles: 6.12, 12.26, 12.44, 13.42, 18.46, and 19.26°;

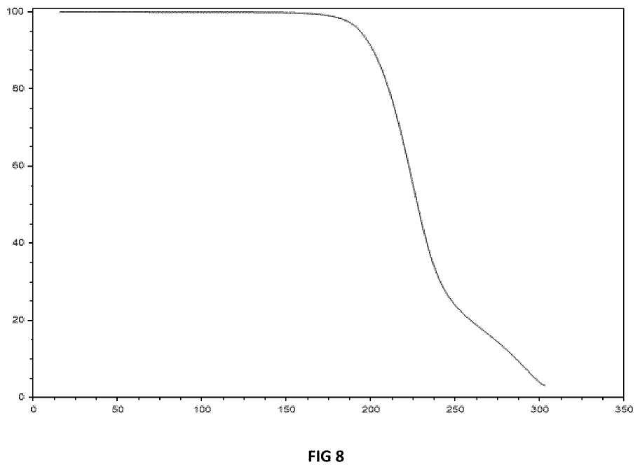

c) a thermo-gravimetric analysis (TGA) substantially similar to the one set forth in FIG. 8; or

d) combination thereof.

E11. The crystalline form according to embodiment E10, wherein said crystalline form has a crystal form characterized by an XRPD obtained using CuK_{α1} radiation (λ=1.5406 Å) showing peaks at the following 2θ-angles: 6.12, 12.26, 12.44, 13.42, 18.46, and 19.26°.

E12. The crystalline form according to embodiment E10, wherein said crystalline form has a crystal form characterized by an XRPD obtained using CuK_{α1} radiation (λ=1.5406 Å) showing an XRPD pattern substantially the same as shown in FIG. 7.

E13. The crystalline form according to embodiment E10, wherein the crystalline form has a thermo-gravimetric analysis (TGA) substantially similar to the one set forth in FIG. 8.

E14. The crystalline form according to embodiment E1, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form E having at least one of the following properties:

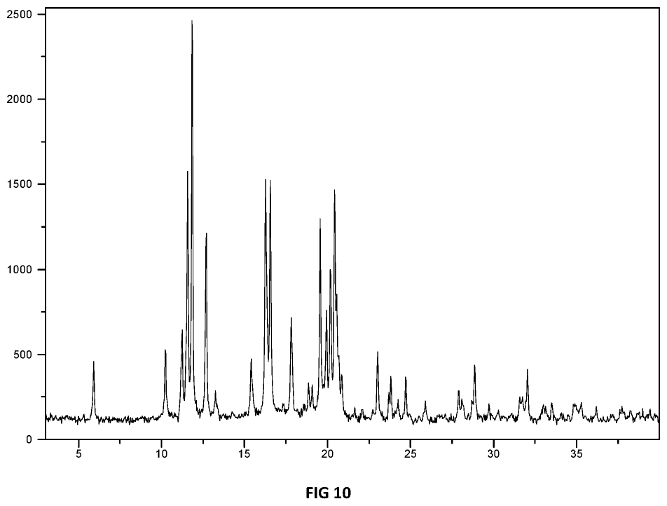

a) an X-ray powder diffraction (XRPD) obtained using CuK_{α1} radiation (λ=1.5406 Å) showing an XRPD pattern substantially the same as shown in FIG. 10;

b) an X-ray powder diffraction (XRPD) pattern obtained using CuK_{α1} radiation (λ=1.5406 Å) showing characteristic peaks at the following 2θ-angles: 10.25, 15.41, 16.29, 16.55, 19.56, and 24.72°;

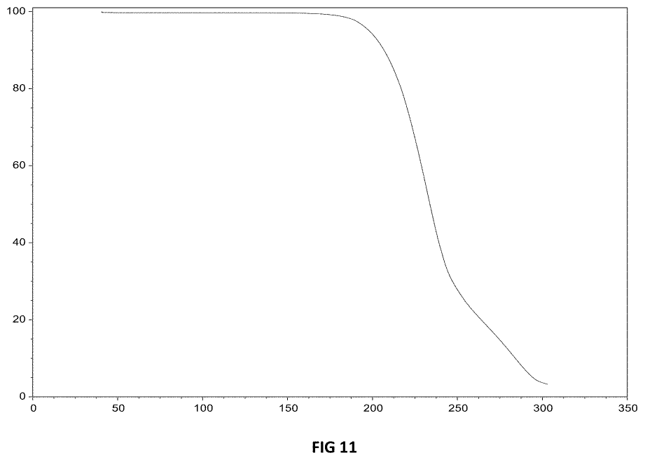

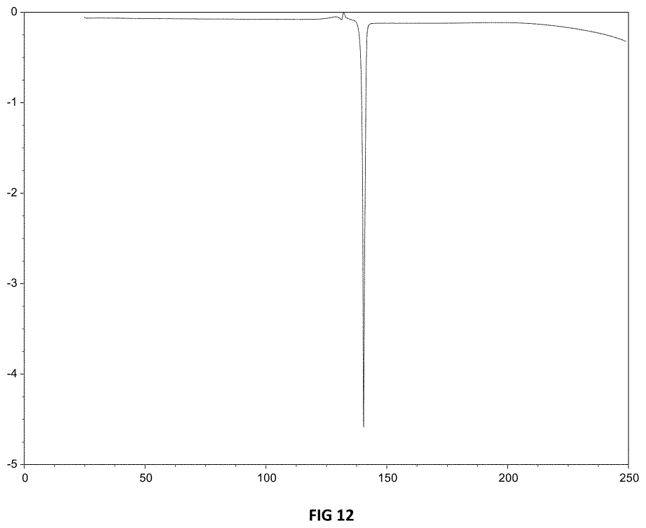

c) a thermo-gravimetric analysis (TGA) substantially similar to the one set forth in FIG. 11; or

d) combination thereof.

E15. The crystalline form according to embodiment E14, wherein said crystalline form has a crystal form characterized by an XRPD obtained using CuK_{α1} radiation (λ=1.5406 Å) showing peaks at the following 2θ-angles: 10.25, 15.41, 16.29, 16.55, 19.56, and 24.72°.

E16. The crystalline form according to embodiment E14, wherein said crystalline form has a crystal form characterized by an XRPD obtained using CuK_{α1} radiation (λ=1.5406 Å) showing an XRPD pattern substantially the same as shown in FIG. 10.

E17. The crystalline form according to embodiment E14, wherein the crystalline form has a thermo-gravimetric analysis (TGA) substantially similar to the one set forth in FIG. 11.

E18. The crystalline form according to embodiment E1, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid hydrate having at least one of the following properties:

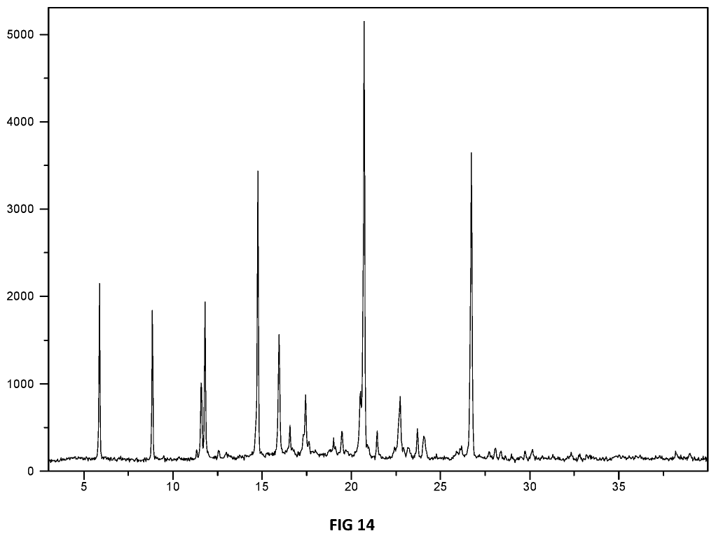

a) an X-ray powder diffraction (XRPD) pattern obtained using CuK_{α1} radiation (λ=1.5406 Å) showing an XRPD pattern substantially the same as shown in FIG. 14;

b) an X-ray powder diffraction (XRPD) pattern obtained using CuK_{α1} radiation (λ=1.5406 Å) showing characteristic peaks at the following 2θ-angles: 8.84, 14.74, 15.95, 17.42, 20.70, and 22.71°;

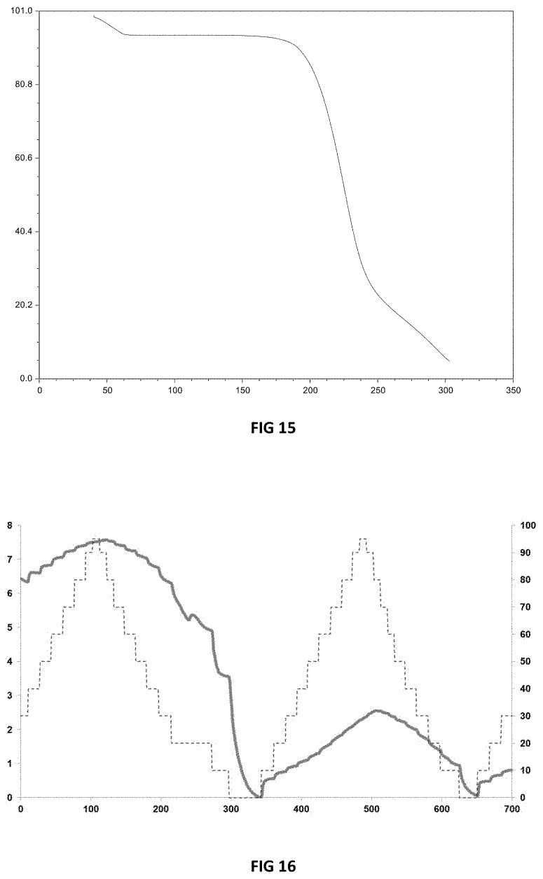

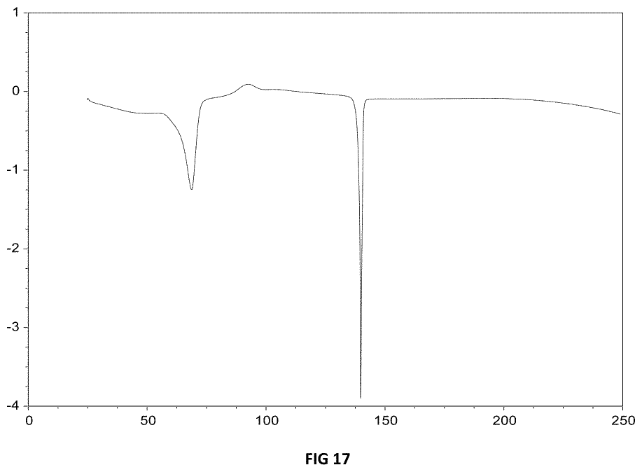

c) a thermo-gravimetric analysis (TGA) substantially similar to the one set forth in FIG. 15; or

d) combination thereof.

E19. The crystalline form according to embodiment E18, wherein said crystalline form has a crystal form characterized by an XRPD obtained using CuK_{α1} radiation (λ=1.5406 Å) showing peaks at the following 2θ-angles: 8.84, 14.74, 15.95, 17.42, 20.70, and 22.71°.

E20. The crystalline form according to embodiment E18, wherein said crystalline form has a crystal form characterized by an XRPD obtained using CuK_{α1} radiation (λ=1.5406 Å) showing an XRPD pattern substantially the same as shown in FIG. 14.

E21. The crystalline form according to embodiment E18, wherein the crystalline form has a thermo-gravimetric analysis (TGA) substantially similar to the one set forth in FIG. 15

E22. The crystalline form according to embodiment E1, wherein crystalline form is an alkali metal salt of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid.

E23. The crystalline form according to embodiment E22, wherein the pharmaceutically acceptable salt is a sodium salt of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid.

E24. The crystalline form according to embodiment E23, wherein said sodium salt is a monohydrate of the sodium salt of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid.

E25. The crystalline from according to embodiment E23 to E24, wherein said sodium salt monohydrate form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid has the following structure:

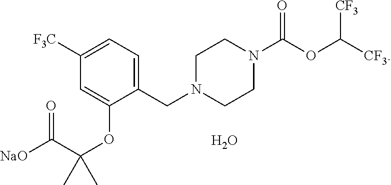

E26. The crystalline form according to embodiment E1 and embodiments E22 to E25, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid sodium salt monohydrate form having at least one of the following properties:

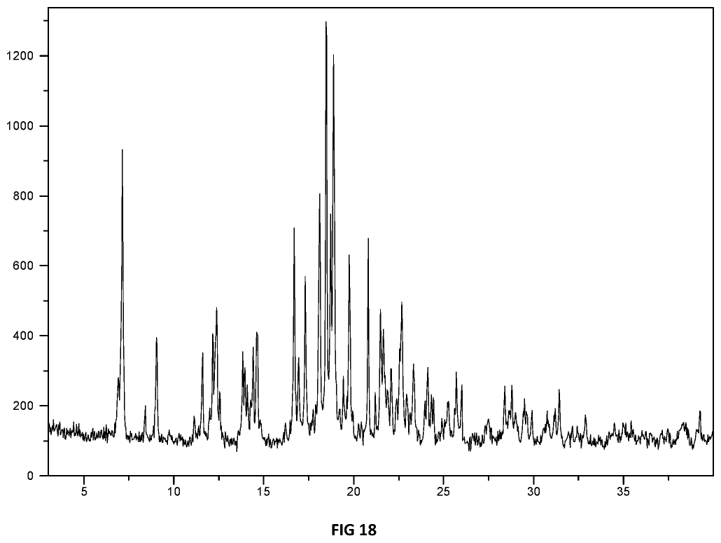

a) an X-ray powder diffraction (XRPD) obtained using CuK_{α1} radiation (λ=1.5406 Å) showing an XRPD pattern substantially the same as shown in FIG. 18;

b) an X-ray powder diffraction (XRPD) pattern obtained using CuK_{α1} radiation (λ=1.5406 Å) showing characteristic peaks at the following 2θ-angles: 7.13, 9.03, 12.16, 18.09, 18.47, and 18.89°;

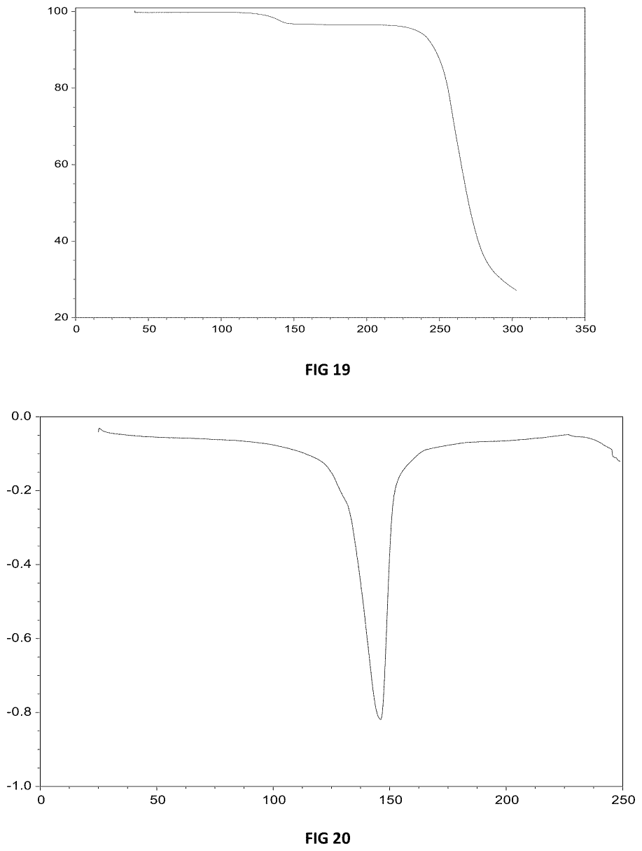

c) a thermo-gravimetric analysis (TGA) substantially similar to the one set forth in FIG. 19;

d) DSC thermogram substantially similar to the one set forth in FIG. 20;

e) DSC thermogram with an endotherm having an onset at about 130° C.; or

f) two or more of a) to e);

E27. The crystalline form according to embodiment E26, wherein said crystalline form has a crystal form characterized by an XRPD obtained using CuK_{α1} radiation (λ=1.5406 Å) showing peaks at the following 2θ-angles: 7.13, 9.03, 12.16, 18.09, 18.47, and 18.89°.

E28. The crystalline form according to embodiment E26, wherein said crystalline form has a crystal form characterized by an XRPD obtained using CuK_{α1} radiation (λ=1.5406 Å) showing an XRPD pattern substantially the same as shown in FIG. 18.

E29. The crystalline form according to embodiment E26, wherein the crystalline form has a thermo-gravimetric analysis (TGA) substantially similar to the one set forth in FIG. 19.

E30. The crystalline form according to embodiment E26, wherein the crystalline form has a DSC thermogram with an endotherm having an onset at about 130° C.

E31. The crystalline form according to embodiments E1 to E30, wherein the crystalline form is substantially pure.

E32. The crystalline form according to embodiments E1 to E31, wherein the purity of the crystalline form is no less than 95%, no less than about 96%, no less than about 97%, no less than about 98%, no less than about 98.5%, no less than about 99%, no less than about 99.5%, or no less than about 99.8%.

E33. A pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E1 to E32 and one or more pharmaceutically acceptable carriers or diluents.

E34. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32, for use as a medicament.

E35. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E1 to E32, for use in the treatment of a disease or a disorder selected from pain, epilepsy/seizure disorder, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

E36. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32 for use in the treatment according to embodiment E35, wherein the disorder is epilepsy/seizure disorder.

E37. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32, for use in the treatment of epilepsy/seizure according to embodiment E36, wherein the epilepsy/seizure is selected from acute repetitive seizures, temporal lobe epilepsy, Dravet syndrome, Lennox Gastaut syndrome and Angelman syndrome.

E38. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32, for use in the treatment of multiple sclerosis.

E39. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32, for use in the treatment of one or more symptoms in multiple sclerosis according to embodiment E38, wherein the one or more symptoms is selected from fatigue, spasticity, depression, behavioral disturbance, irritability-agitation, and pain.

E40. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32, for use in the treatment of pain according to embodiment E35, wherein the disorder is pain selected from acute pain, neuropathic pain, inflammatory pain, cancer pain, chronic pain, pain caused by peripheral neuropathy, central pain, complex regional pain syndrome, fibromyalgia, migraine, vasoocclussive painful crises in sickle cell disease, spasticity or pain associated with multiple sclerosis, abdominal pain associated with irritable bowel syndrome functional chest pain, rheumatoid arthritis, osteoarthritis, somatoform disorders, or functional dyspepsia.

E41. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E1 to E32, for use in the treatment of pain according to embodiment E40, wherein the pain is neuropathic pain.

E42. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E1 to E32, for use in the treatment of neuropathic pain according to embodiment E41, wherein the neuropathic pain is selected from neuropathic pain in spinal cord injury and neuropathic pain in cervical dystonia.

E43. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E1 to E32, for use in the treatment pain according to E40, wherein the pain is central pain.

E44. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E1 to E32, for use in the treatment pain according to E40, wherein the pain is fibromyalgia.

E45. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E1 to E32, for use in the treatment pain according to E40, wherein the pain is migraine.

E46. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32, for use in the treatment of migraine according to embodiment E45, wherein the migraine is selected from prevention of chronic migraine and hemiplegic migraine.

E47. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E1 to E32, for use in the treatment of disorders according to embodiment E35, wherein the disorder is selected from multiple sclerosis, Parkinson's disease, Huntington's disease, Alzheimer's disease, and amyotrophic lateral sclerosis.

E48. A method for the treatment of disease or a disorder selected from pain, epilepsy/seizure disorder, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia; which method comprises the administration of a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32, to a patient in need thereof.

E49. Use of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E1 to E32, in the manufacture of a medicament for the treatment of disease or a disorder selected from pain, epilepsy/seizure disorder, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

E50. A crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof.

E51. The crystalline form according to embodiment E50, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form A having a crystal form characterized by an XRPD obtained using CuK_{α1} radiation (λ=1.5406 Å) showing peaks at the following 2θ-angles: 10.36, 16.45, 16.71, 19.66, 21.85, and 24.93°.

E52. The crystalline form according to embodiment E50, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form C having a crystal form characterized by an XRPD obtained using CuK_{α1} radiation (λ=1.5406 Å) showing peaks at the following 2θ-angles: 5.75, 11.08, 12.50, 15.21, 17.58, and 20.06°.

E53. The crystalline form according to embodiment E50, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form D having a crystal form characterized by an XRPD obtained using CuK_{α1} radiation (λ=1.5406 Å) showing peaks at the following 2θ-angles: 6.12, 12.26, 12.44, 13.42, 18.46, 19.26°.

E54. The crystalline form according to embodiment E50, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form E having a crystal form characterized by an XRPD obtained using CuK_{α1} radiation (λ=1.5406 Å) showing peaks at the following 2θ-angles: 10.25, 15.41, 16.29, 16.55, 19.56, and 24.72°.

E55. The crystalline form according to embodiment E50, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid hydrate having a crystal form characterized by an XRPD obtained using CuK_{α1} radiation (λ=1.5406 Å) showing peaks at the following 2θ-angles: 8.84, 14.74, 15.95, 17.42, 20.70, and 22.71°.

E56. The crystalline form according to embodiment E50, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid sodium salt monohydrate form having a crystal form characterized by an XRPD obtained using CuK_{α1} radiation (λ=1.5406 Å) showing peaks at the following 2θ-angles: 7.13, 9.03, 12.16, 18.09, 18.47, and 18.89°.

E57. A pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, according to any of embodiments E50 to E56 and one or more pharmaceutically acceptable carriers or diluents.

E58. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E50 to E56, for use as a medicament.

E59. The crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt, according to any of embodiments E50 to E56, for use in the treatment of a disease or a disorder selected from pain, epilepsy/seizure disorder, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

E60. A method for the treatment of disease or a disorder selected from pain, epilepsy/seizure disorder, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia; which method comprises the administration of a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E50 to E56, to a patient in need thereof.

E61. Use of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof according to any of embodiments E50 to E56, in the manufacture of a medicament for the treatment of disease or a disorder selected from pain, epilepsy/seizure disorder, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

### Pharmaceutical Compositions

The above-mentioned compounds or pharmaceutically acceptable salts may be in a composition as the sole active ingredient or in combination with other active ingredients. Additionally, one or more pharmaceutically acceptable carriers or diluents may be in the composition.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragées, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include suppositories, sprays, ointments, creams, gels, inhalants, dermal patches, implants, etc.

Conveniently, the compounds of the invention are administered in a unit dosage form containing said compounds in an amount of about 0.1 to 500 mg, such as 1 mg, 2 mg, 4 mg, 6 mg, 8 mg, 10 mg, 20 mg, 30 mg, 40 mg, 50 mg 100 mg, 150 mg, 200 mg or 250 mg of a compound of the present invention.

For parenteral administration, solutions of the compound of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phosphor lipids, fatty acids, fatty acid amines, polyoxyethylene and water. The pharmaceutical compositions formed by combining the compound of the invention and the pharmaceutically acceptable carriers are then readily administered in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by compression of the mixture in a conventional tabletting machine. Examples of adjuvants or diluents comprise corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

### Conditions for Treatment

The compounds of the present invention are intended for treatment of diseases and disorders which are linked to regulation of endocannabinoid system signaling activities where a MAGL inhibitor may be therapeutically beneficial. As described above the compounds of the invention may be beneficial in indication whose pathological hallmarks include excessive neurotransmission, neuroinflammation or neurodegeneration.

Hence, in an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use as a medicament.

In a further embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid or pharmaceutically acceptable salt thereof, for use in the treatment of a disease or a disorder selected from pain, epilepsy/seizure disorder, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

### Epilepsy and Seizure Treatment

In a study by Sugaya et al., Cell Rep. 2016, it is suggested 2-AG is crucial for suppressing seizures. Hence, in another embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of a epilepsy/seizure disorder. Furthermore, it has been suggested by Yeh et al., Perspectives on the Role of Endocannabinoids in Autism Spectrum Disorders, OBM Neurobiol. 2017. that targeting the endocannobinoid signaling is a potential way forward for treating symptoms within Autism spectrum disorders.

In a further embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of acute repetitive seizures, temporal lobe epilepsy, Dravet syndrome, Lennox Gastaut syndrome or Angelman syndrome.

### Pain Treatments

In an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of pain selected from acute pain, inflammatory pain, cancer pain, chronic pain, pain caused by peripheral neuropathy, central pain, complex regional pain syndrome, fibromyalgia, migraine, vasoocclussive painful crises in sickle cell disease, spasticity or pain associated with multiple sclerosis, abdominal pain associated with irritable bowel syndrome functional, chest pain, rheumatoid arthritis, osteoarthritis, somatoform disorders, or functional dyspepsia.

In an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic, acid or pharmaceutically acceptable salt thereof, for use in the treatment of neuropathic pain.

In an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of neuropathic pain selected from neuropathic pain in spinal cord injury and neuropathic pain in cervical dystonia.

### Central Pain

Central pain is neuropathic pain caused by lesion or dysfunction of the central nervous system, for example, post-stroke, multiple sclerosis, neuromyelitis optica, idiopathic inflammatory transverse myelitis, spinal cord injury, brachial-radial pain syndrome, and central craniofacial pain. Exocannabinoids have demonstrated activity in central pain associated with multiple sclerosis. A 4-week randomized double-blind placebo-controlled parallel group trial with MS and central pain using an oromucosal spray, THC/CBD, containing the CB₁ agonist delta-9-tetrahydrocannabinol and cannabidiol (another Cannabis-derived alcohol) showed that the active agent was superior to placebo in reducing the mean intensity of pain (NRS-11) and of sleep disturbance (Rog et al., Neurology. 2005). The same THC/CBD preparation was studied in a larger group of MS patients with central neuropathic pain utilizing a two-stage design; in the second phase of this study, the time to treatment failure (primary endpoint) statistically favored THC/CBD, as did an improvement in the Pain NRS-11 and sleep quality (Langford et al., J Neurol. 2013). Additionally, nabilone, a synthetic CB₁ agonist structurally related to THC, showed efficacy in MS-induced central neuropathic pain (Turcotte et al., Pain Med. 2015). Studies of exocannabinoids in central pain have indicated activity, suggesting MAGL inhibitors may also have efficacy in treatment of central pain. Hence, in an embodiment, disclosed herein, is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of central pain.

### Fibromyalgia

Fibromyalgia (FM) is a common, chronic, idiopathic condition characterized by diffuse body pain and the presence of pressure allodynia. Duloxetine and pregabalin are specifically labeled for the treatment of pain in FM, and tricyclic antidepressants like amitriptyline, while not specifically labeled for FM treatment, are first-line agents. There is no clear pathological understanding of FM, and no validated preclinical model. However, studies of exocannabinoids in FM have indicated activity, suggesting MAGL inhibitors may also have efficacy in treatment of FM. Measures of pain (e.g., NRS-11, Pain VAS) and the Fibromyalgia Impact Questionnaire (FIQ), which measures limitations in several activities of daily living impacted by FM, have demonstrated activity of drugs in FM clinical trials (Burckhardt et al., J Rheumatol. 1991); Mease et al., J Rheumatol. 2008). A survey of Spanish FM patients who were cannabis users and non-users was performed to identify the effects of cannabis on a range of disease symptoms such as pain, stiffness, well-being, relaxation and drowsiness; perceived relief was common for pain, sleep disturbances, stiffness mood disorders and anxiety (Fiz, PLoS One, 2011, 6(4), e18440). In an 8-week, 40-patient study, compared with placebo the exocannabinoid nabilone improved pain measured on a 10 cm VAS, and improved the FIQ domain of anxiety and the FIQ total score (Skrabek et al., J Pain. 2008). In a 31-patient study, compared with amitriptyline nabilone improved the index of sleep (Insomnia Severity Index) and was judged non-inferior on measures of pain (McGill Pain Questionnaire) and the FIQ (Ware, Anesth Analg, 2010, 110(2), 604-10). Hence, in an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of fibromyalgia.

### Migraine

Migraine is a common episodic disorder of head and facial pain. Migraine attacks can be acutely treated with NSAIDs, acetaminophen, a variety of triptans (e.g., sumatriptan), and antiemetics, but some migraine sufferers have pain unresponsive to existing treatment options. Data suggests that endocannabinoid pathways may be relevant in migraine. In patients with chronic migraine and probable analgesic-overuse headache, CSF samples showed higher levels of the endocannabinoid palmitoylethanolamide and lower levels of anandamide compared with healthy controls (Sarchielli et al., Neuropsychopharmacology. 2007). In addition, a retrospective chart review of patients attending a medical marijuana clinic with a primary diagnosis of migraine headaches found a decrease in the frequency of migraine headaches after initiating marijuana therapy (Rhyne et al., Pharmacotherapy. 2016), suggesting MAGL inhibitors may also have efficacy in treatment of migraine. Hence, in an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of migraine.

In a further embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of prevention of chronic migraine and hemiplegic migraine.

### Neurodegenerative Disorders

In an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of disorders selected from multiple sclerosis, Parkinson's disease, Huntington's disease, Alzheimer's disease, and amyotrophic lateral sclerosis.

### Multiple Sclerosis Symptomatic Treatment

Nearly all MS patients of all subtypes have one or more symptoms of spasticity, pain, disturbed sleep, bladder dysfunction, and fatigue. Disease modifying therapies do not improve symptoms. Spasticity affects over 80% of MS patients; 34% have moderate, severe, or total spasticity. Severe spasticity is related to cost and level of care, and is independently related to quality of life in MS. Two recent reviews support the use of exocannabinoids for the treatment of MS spasticity and pain (Whiting et al., JAMA. 2015); Hill et al., JAMA. 2015).

An exocannabinoid preparation is an approved treatment for spasticity associated with MS. Sativex, an oromucosal spray mixture of the CB₁ agonist THC and another cannabis plant derived alcohol, cannabidiol, was shown to decrease self-reported spasticity related symptoms. In a pivotal trial of Sativex using a randomized withdrawal design, there was improvement with continuing Sativex in spasm frequency, sleep disruption by spasticity, subject global impression of change, carer global impression of change, and physician global impression of change. Other clinical trials have shown activity of a variety of exocannabinoids in spasticity due to MS (Zajicek et al., Lancet. 2003; Collin et al., Eur J Neurol. 2007; Collin et al., Neurol Res. 2010). These parallel group studies exemplify the clinical trial design and endpoints that could be used to show a MAGL benefits spasticity in MS.

In an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of multiple sclerosis.

In an embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of one or more symptoms in multiple sclerosis selected from fatigue, spasticity, depression, behavioral disturbance, irritability-agitation, and pain.

It is believed that a MAGL will also be beneficial in the treatment of indications related to autoimmune encephalomyelitis. Hence, in a further embodiment, disclosed herein is a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of Rasmussen encephalitis, Systemic lupus erythematosus, Behcet's disease, Hashimoto's encephalopathy, and Sydenham's chorea.

### Amyotrophic Lateral Sclerosis

In Pryce et al. Handb Exp Pharmacol. 2015; 231: 213-31 it is described how Patients of Amyotrophic Lateral Sclerosis (ALS) typically experience muscle weakness and/or fasciculations which gradually worsen, bulbar symptoms and eventually respiratory problems. In preclinical model of ALS it has been suggested that cannabinoids may have a significant neuroprotective effect.

Hence, in an embodiment disclosed herein is provided a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of amyotrophic lateral sclerosis.

### Mood and Anxiety Disorders

Mood and anxiety disorders are chronic, disabling conditions that impose cost to both patients and society. In relation to mood and anxiety disorders, the endocannabinoid system has received increasingly more intention in recent years. A recent study by Bedse G et al., Transl Psychiatry. 2018 it is suggested that the use of MAGL inhibitors may have a beneficial effect in stress-related psychopathology. Hence, in an embodiment, disclosed herein, a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of mood and anxiety disorders.

In a further embodiment, disclosed herein, a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of mood and anxiety disorders selected from depression, and GAD.

In a further embodiment disclosed herein, a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of depression selected from major depressive disorder, treatment-resistant depression, catatonic depression, melancholic depression, atypical depression, psychotic depression, perinatal depression, postpartum depression, bipolar depression, including bipolar I depression and bipolar II depression, and mild, moderate or severe depression. In a further embodiment disclosed herein, a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of major depressive disorder.

In a further embodiment, disclosed herein, a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of GAD.

### Post-Traumatic Stress Disorder

Post-traumatic stress disorder (PTSD is a trauma- or stress-related disorder. Patients having PTSD will have the symptoms of flashback of trauma, avoidance, hyperarousal, and negative cognitions/moods. In a review by Hill et al., Neuropsychopharmacology. 2018 it is suggested that drugs that affect the endocannabinoid signaling (such as MAGL inhibitors) may be beneficial in treating symptoms of PTSD. Hence, in an embodiment, disclosed herein, a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or pharmaceutically acceptable salt thereof, for use in the treatment of PTSD.

In some embodiments disclosed herein are methods of modulating the activity of MAGL. Contemplated methods, for example, comprise exposing said enzyme to a compound described herein. The ability of compounds described herein to modulate or inhibit MAGL is evaluated by procedures known in the art and/or described herein. Another aspect of this disclosure provides methods of treating a disease associated with expression or activity of MAGL in a patient.

### Combination Therapies

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is affected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

Combination therapy also embraces the administration of the therapeutic agents as described above in further combination with other biologically active ingredients and non-drug therapies. Where the combination therapy further comprises a non-drug treatment, the non-drug treatment is conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as, conventional oral dosage forms, that are administered either simultaneously or sequentially.

For example, for contemplated treatment of pain, a disclosed compound is co-administered with another therapeutic for pain such as an opioid, a cannabinoid receptor (CB₁ or CB₂) modulator, a COX-2 inhibitor, acetaminophen, and/or a non-steroidal anti-inflammatory agent. Additional therapeutics e.g., for the treatment of pain that are co-administered, include morphine, pregabalin, gabapentin, codeine, hydromorphone, hydrocodone, oxymorphone, fentanyl, tramadol, and levorphanol.

Other contemplated therapeutics for co-administration include aspirin, naproxen, ibuprofen, salsalate, diflunisal, dexibuprofen, fenoprofen, ketoprofen, oxaprozin, loxoprofen, indomethacin, tolmetin, sulindac, etodolac, ketorolac, piroxicam, meloxicam, tenoxicam, droxicam, lornoxicam, celecoxib, parecoxib, rimonabant, and/or etoricoxib.

### Item List

In a further aspect described herein is provided treatment of disorders or diseases with 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof. In the following, embodiments of said aspect are disclosed. The first embodiment is denoted EE1, the second embodiment is denoted EE2 and so forth.

EE1. 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment of a disorder selected from acute repetitive seizures, Lennox Gastaut syndrome Angelman syndrome, temporal lobe epilepsy, Dravet syndrome, post-traumatic stress disorder, depression, and generalized anxiety disorder.

EE2. 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment according to embodiment EE1, wherein said disorder is selected from Angelman syndrome, Lennox Gastaut syndrome, acute repetitive seizures, temporal lobe epilepsy, and Dravet syndrome.

EE3. 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment according to embodiment EE1, wherein said disorder is selected from PTSD, depression, and GAD.

EE4. 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment according to embodiment EE3, wherein said disorder is PTSD.

EE6. 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment according to embodiment EE3, wherein said disorder is generalized anxiety disorder.

EE7. 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment according to embodiment EE3, wherein said disorder is depression.

EE8. 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment according to embodiment EE7, wherein the depression is selected from major depressive disorder, treatment-resistant depression, catatonic depression, melancholic depression, atypical depression, psychotic depression, perinatal depression, postpartum depression, bipolar depression, including bipolar I depression and bipolar II depression, and mild, moderate or severe depression.

EE9. 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt thereof, for use in the treatment according to embodiment EE8, wherein the depression is major depressive disorder.

EE10. A method for the treatment of disease or a disorder selected from acute repetitive seizures, Angelman syndrome, Lennox Gastaut syndrome, temporal lobe epilepsy, Dravet syndrome, post-traumatic stress disorder, depression, generalized anxiety disorder, and somatoform disorders; which method comprises the administration of a therapeutically effective amount of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, to a patient in need thereof.

EE11. Use of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, in the manufacture of a medicament for the treatment of disease or a disorder selected from acute repetitive seizures, Angelman syndrome, Lennox Gastaut syndrome, temporal lobe epilepsy, Dravet syndrome, post-traumatic stress disorder, depression, generalized anxiety disorder, and somatoform disorders.

### Experimental Section

### List of Abbreviations

As used above, and throughout the description of the invention, the following abbreviations, unless otherwise indicated, shall be understood to have the following meanings:
t-Bu: tert-butyl
DCM: dichloromethane (CH₂Cl₂)
DMF: dimethylformamide
DMSO: dimethylsulfoxide
equiv: equivalent(s)
EtOH: ethanol
EtOAc: ethyl acetate
HPLC: high performance liquid chromatography
i-PrOAc: isopropyl acetate
MS: mass spectroscopy
NMR: nuclear magnetic resonance
MTBE: Tert-Butyl Methyl Ether
TFA: trifluoroacetic acid
TLC: thin layer chromatography
RT: room temperature

### Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1. Preparation of Compound (I)

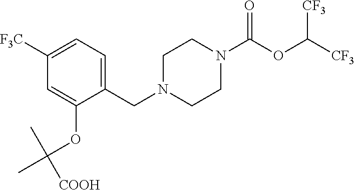 [SMILES: CC(C)(Oc1cc(C(F)(F)F)ccc1CN1CCN(C(=O)OC(C(F)(F)F)C(F)(F)F)CC1)C(=O)O]

Step 1: Preparation of t-butyl 2-(2-formyl-5-(trifluoromethyl)phenoxy)-2-methylpropanoate

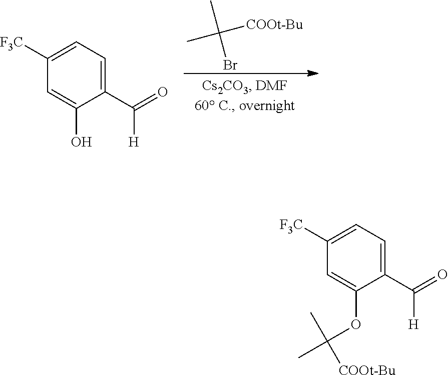

A round-bottom flask was charged with 2-hydroxy-4-(trifluoromethyl)benzaldehyde (0.200 g, 1.05 mmol, 1.00 equiv), tert-butyl 2-bromo-2-methylpropanoate (0.466 g, 2.09 mmol, 2.00 equiv), cesium carbonate (1.03 g, 3.16 mmol, 3.00 equiv), and DMF (5 mL). The reaction mixture was stirred overnight at 60° C. and quenched with water (20 mL). The resulting solution was extracted with DCM (3×20 mL) and the organic layers were combined, washed with brine (20 mL), dried over anhydrous Na₂SO₄, filtered and concentrated under reduced pressure. The residue was chromatographed to provide 0.150 g (52% yield) of t-butyl 2-(2-formyl-5-(trifluoromethyl)phenoxy)-2-methylpropanoate as a yellow oil. LCMS (ESI, m/z): 333 [M+H]⁺.

### Step 2: Preparation of 1,1,1,3,3,3-hexafluoropropan-2-yl 4-(2-((1-(tert-butoxy)-2-methyl-1-oxopropan-2-yl)oxy)-4-(trifluoromethyl)benzyl)piperazine-1-carboxylate

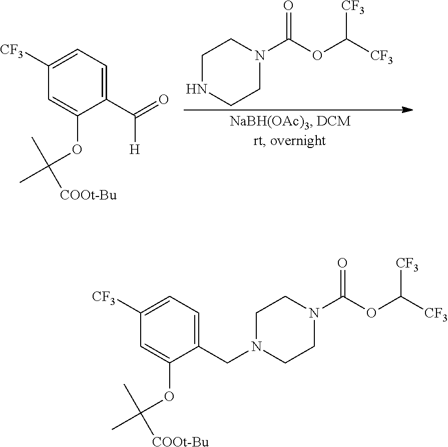

A round-bottom flask was charged with t-butyl 2-(2-formyl-5-(trifluoromethyl)phenoxy)-2-methylpropanoate (200 mg, 0.600 mmol, 1.00 equiv), 1,1,1,3,3,3-hexafluoropropan-2-yl piperazine-1-carboxylate (202 mg, 0.720 mmol, 1.20 equiv), and DCM (10 mL). The mixture was stirred for 1 h at room temperature prior to addition of sodium triacetoxyborohydride (511 mg, 2.41 mmol, 4.00 equiv). The reaction mixture was stirred overnight at room temperature and quenched with water (20 mL). The resulting solution was extracted with DCM (3×20 mL) and the organic layers were combined, washed with brine (30 mL), dried over anhydrous Na₂SO₄, filtered and concentrated under reduced pressure. The residue was chromatographed on a silica gel column to provide 300 mg (84% yield) of 1,1,1,3,3,3-hexafluoropropan-2-yl 4-(2-((1-(t-butoxy)-2-methyl-1-oxopropan-2-yl)oxy)-4-(trifluoromethyl)benzyl)piperazine-1-carboxylate as a yellow oil. LCMS (ESI, m/z): 597 [M+H]⁺.

### Step 3: Preparation of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid

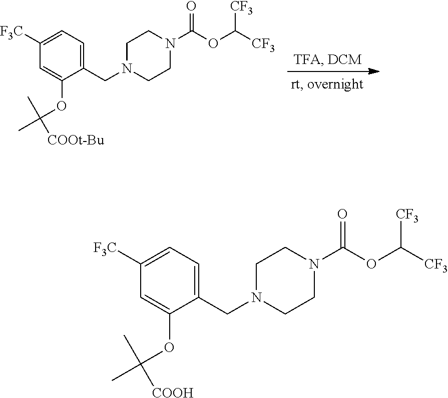

A round-bottom flask was charged with 1,1,1,3,3,3-hexafluoropropan-2-yl 4-(2-((1-(t-butoxy)-2-methyl-1-oxopropan-2-yl)oxy)-4-(trifluoromethyl)benzyl)piperazine-1-carboxylate (200 mg, 0.335 mmol, 1.00 equiv), TFA (3 mL), and DCM (10 mL). The resulting solution was stirred overnight at room temperature and concentrated under reduced pressure. The crude product was dissolved in saturated NaHCO₃ solution (10 mL) and extracted with DCM (3×20 mL). The organic layers were combined, washed with brine (30 mL), dried over anhydrous Na₂SO₄, filtered and concentrated under reduced pressure. The crude product (200 mg) was purified by preparative HPLC to provide 60.9 mg (34% yield) of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid as a white solid. ¹H NMR (300 MHz, Methanol-d₄) δ 7.54 (d, J=7.8 Hz, 1H), 7.23-7.19 (m, 2H), 6.18-6.10 (m, 1H), 3.80 (s, 2H), 3.63 (s, 4H), 2.71 (s, 4H), 1.60 (s, 6H). LCMS (ESI, m/z): 541 [M+H]⁺.

### Example 2. Preparation of Compound (I) Form A

Compound (I) form A was obtained using the following procedure.

Compound (I) (4.23 g) in i-PrOAc (15 mL) heated to 80° C. to dissolve Compound (I). Heptane (32 mL) was added dropwise at 80° C. The solution was allowed to cool to 50° C. Additional heptane (45 mL) was added dropwise over 40 min at 50° C. and precipitation formed. The slurry was allowed to cool to room temperature overnight and then cooled in ice-bath for 1.5 hours before filtering. The filter cake was washed with i-PrOAc:heptane, 1:4 (8 mL) and dried in vacuum oven at 40° C., yielding the product as a white solid (3.33 g). NMR complies with structure.

### Example 3. Preparation of Compound (I) Form C

Compound (I) form C was obtained using the following procedure:

Compound (I) 133 mg was added 700 μL isopropylacetate and heated to 80° C. whereby it dissolved. The solution was cooled to RT and precipitation occurred within 12 to 20 h. The solid was left at RT for 2 days before the slurry was placed in open for evaporation to dry. NMR complies with structure.

### Example 4. Preparation of Compound (I) Form D

Compound (I) form D was obtained using the following procedure.

Compound (I) (1.08 g) in EtOAc (3 mL) was heated to reflux. Heptane (6 mL) added dropwise to the solution while heating to 80° C. The solution was allowed to cool to 55° C. Heptane (9 mL) was added dropwise to the solution at 55° C. and precipitation formed. The mixture was quickly cooled in ice bath, filtered and washed with ambient temperature heptane. The filter cake was allowed to dry in fume hood over the weekend yielding the product as a white powder (0.88 g). NMR complies with structure.

### Example 5. Preparation of Compound (I) Form E

Compound (I) form E was obtained using the following procedure.

Compound (I) form A (3.00 g) in i-PrOAc (15 mL) and heptane (15 mL) was stirred at ambient temperature for 3 days before filtering and washing with heptane. The white solid was dried at ambient temperature in the fume hood. NMR complies with structure.

### Example 6. Preparation of Compound (I) Hydrate

Compound (I) hydrate was obtained using the following procedure.

Compound (I) (0.32 g) was stirred in water (1.5 mL) and EtOH (1 mL) over the weekend at ambient temperature, filtered and allowed to dry in the fume hood. NMR complies with structure.

### Example 7. Preparation of Compound (I) Na.H₂O

Compound (I) Na.H₂O was obtained using the following procedure. Compound (I) (5.10 kg) was added to ethanol (8.16 kg) at room temperature under stirring. The solution was heated to around 50° C. Sodium hydroxide (27%, 0.99 equiv.) was added under stirring followed by addition of 2-Propanol (8.14 kg) over 20-45 min at around 50° C. The solution was stirred for an additional period of 2-30 min, followed by addition of MTBE (45.10 kg) over 80-120 min at around 50° C. The solution was then stirred for an additional 25 to 45 min at around 50° C., followed by cooling of the solution to room temperature. The solution was stirred for an additional 12 to 20 hours before cooling the solution to around 0° C. The solution was then stirred for around an hour. The solids were transferred to centrifugation and washed with a solution of ethanol (1.19 kg), 2-propanol (1.2 kg), and MTBE (7.29 kg), followed by an additional wash with MTBE (9.45 kg). The collected solids were dried under vacuum at around 40° C. for around 16 h to give Compound (I) Na.H₂O (4.41 kg). NMR complies with structure.

### Example 8: X-ray Powder Diffraction (XRPD)

X-Ray powder diffractograms were measured on a PANalytical X'Pert PRO X-Ray Diffractometer using CuK_{α1} radiation (λ=1.5406 Å). The samples were measured in reflection mode in the 2θ-range 3-40 using an X'celerator detector.

In table 1 is listed the characteristic peaks measured at λ=1.5406 Å for Compound (I) form A, Compound (I) form C, Compound (I) form D, Compound (I) form E, Compound (I) hydrate, and Compound (I) Na.H₂O. Diffraction data are indicated ±0.05° 2θ.

TABLE 1
Characteristic XRPD peaks of the compounds of the invention

| | Peaks expressed in degree of diffraction angle |
|---|---|
| Crystalline form | [°2θ] |
| Compound (I) form A | 10.36, 16.45, 16.71, 19.66, 21.85, 24.93 |
| Compound (I) form C | 5.75, 11.08, 12.50, 15.21, 17.58, 20.06 |
| Compound (I) form D | 6.12, 12.26, 12.44, 13.42, 18.46, 19.26 |
| Compound (I) form E | 10.25, 15.41, 16.29, 16.55, 19.56, 24.72 |
| Compound (I) hydrate | 8.84, 14.74, 15.95, 17.42, 20.70, 22.71 |
| Compound (I) Na•H₂O | 7.13, 9.03, 12.16, 18.09, 18.47, 18.89 |

XRPD analysis (FIG. 1) of Compound (I) form A showed the form to be crystalline.

XRPD analysis (FIG. 4) of Compound (I) form C showed the form to be crystalline.

XRPD analysis (FIG. 7) of Compound (I) form D showed the form to be crystalline.

XRPD analysis (FIG. 10) of Compound (I) form E showed the form to be crystalline.

XRPD analysis (FIG. 14) of Compound (I) hydrate showed the hydrate to be crystalline.

XRPD analysis (FIG. 18) of Compound (I) Na.H₂O showed the salt to be crystalline.

### Example 9: Thermo-Gravimetric Analysis (TGA)

Thermo gravimetric analysis (TGA) was measured using a TA-instruments Discovery TGA. 1-10 mg sample is heated 10°/min in an open pan under nitrogen flow.

### Example 10: Differential Scanning Calorimetry (DSC)

Differential Scanning Calorimetry was measured using a TA-Instruments Discovery-DSC calibrated at 5°/min to give the melting point as onset value. About 2 mg of sample is heated 5°/min under nitrogen flow in a closed pan with a pinhole in the lid.

DSC analysis of Compound (I) Na.H₂O (FIG. 20) showed a broad endotherm having an onset at about 130° C.

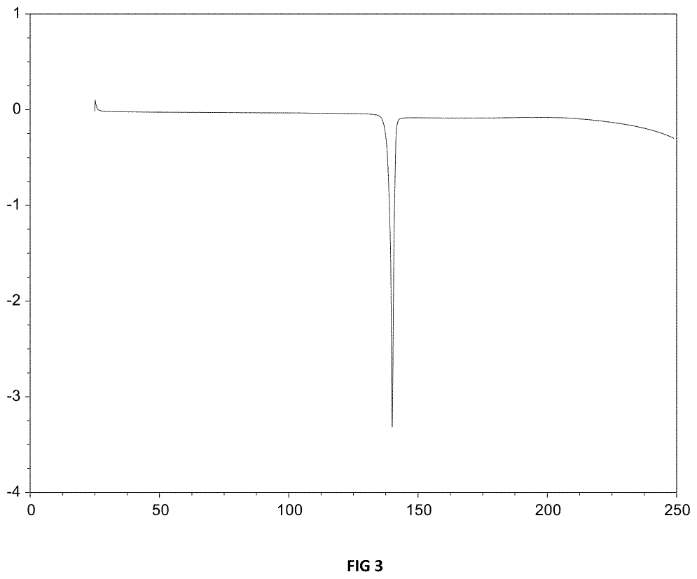

DSC analysis of Compound (I) Form A (FIG. 3) showed an endotherm with onset at 139.2° C. The enthalpy of fusion was 69 J/g.

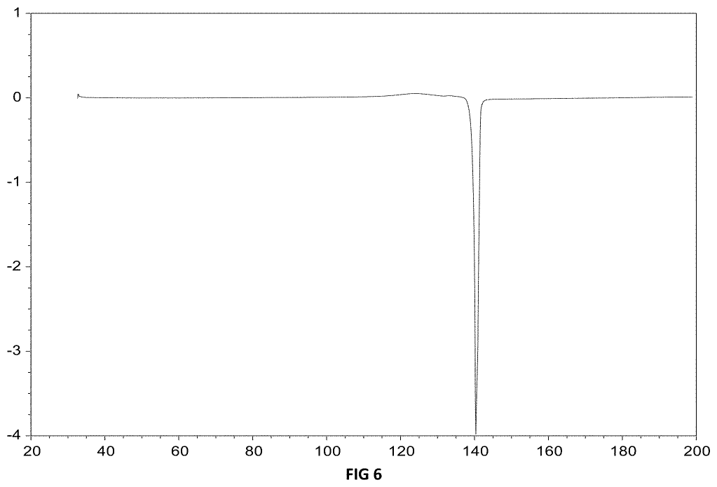

DSC analysis of Form C is shown in FIG. 6. As appears from the thermogram, a broad weak exotherm appears prior to the melting endotherm. XRPD on a sample of Compound (I) Form C heated to 130° C. (that is above the exotherm) showed that it had transformed into Form A. This means that the exothermic transformation of Compound (I) Form C into Compound (I) Form A occurs prior to melting, thus Compound (I) Form A is thermodynamically more stable than Compound (I) Form C.

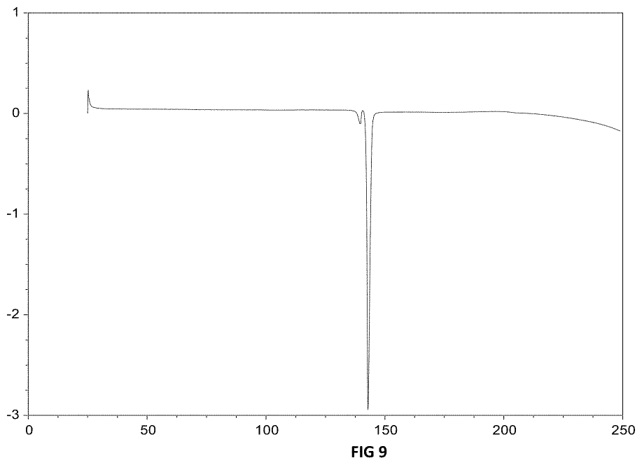

DSC analysis of Compound (I) Form D showed an endotherm having an onset temperature at 142.2° C. and an enthalpy of fusion of 56 J/g. As appears from FIG. 9, Compound (I) Form D has a higher melting point than Compound (I) Form A, but a lower enthalpy of fusion indicating enantiotropic relationship. This means that Compound (I) Form D is more thermodynamically stable at high temperature but less stable at low temperature compared to Compound (I) Form A which is relevant for the further pharmaceutical development and storage stability. Slurry experiments using Compound (I) Form A in different solvents at temperatures up to 60° C. did not lead to transformation into Compound (I) Form D, thus the temperature above which Compound (I) Form D is the more stable form is 60° C. or higher.

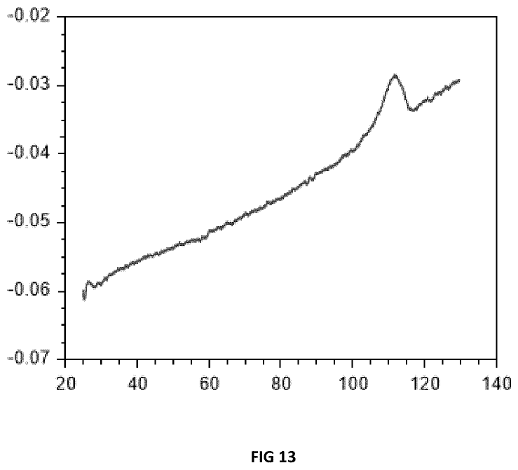

DSC analysis of Form E heated 1° C./min to 130° C. is shown FIG. 13. The curve shows an exothermic event. After the heating, XRPD showed that the sample had transformed into Compound (I) Form A. This means that exothermic transformation of Compound (I) Form E into Compound (I) Form A occurs prior to melting. Thus, Compound (I) Form A is thermodynamically more stable than Compound (I) Form E.

### Example 11—Dynamic Vapor Sorption (DVS)

Dynamic vapour sorption (DVS) was measured using SMS DVS advantage 01 changing the relative humidity from 0% RH to 95% RH in steps of 10% RH (5% between 90 and 95% RH). 2 cycles were performed using 4 mg, starting at 30% RH. The weight changes during the sorption/desorption cycles were plotted, allowing for the hygroscopic nature of the sample to be determined. The DVS curve FIG. 16 show that dehydration of the hydrate occurs at low relative humidity. After the measurement, the sample had turned amorphous.

### Example 12—¹H NMR of Crystalline Forms of Compound (I)

¹H NMR spectra are recorded at 600.16 MHz on a Bruker Avance-III-600 instrument equipped with a TCI CryoProbe. Dimethyl sulfoxide (99.8% D) is used as solvent, and tetramethylsilane (TMS) is used as internal reference standard.

### Example 13—Polarization Microscopy of Crystalline Forms of Compound (I)

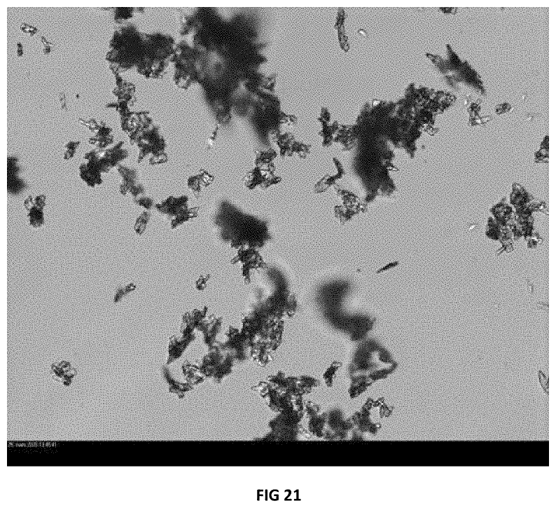

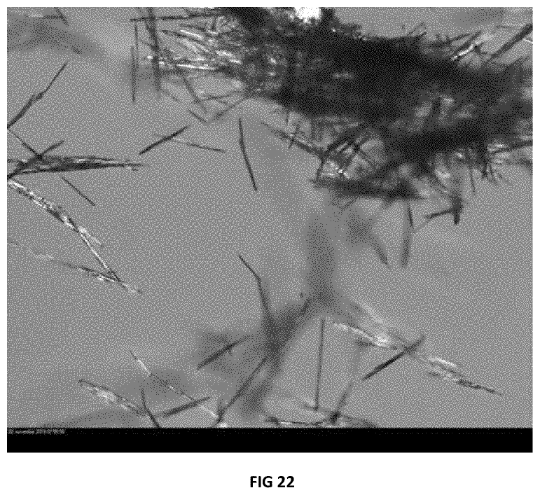

The samples were placed on a microscope slide on a Olympus BX50 polarization microscope equipped with a Pixelink PL-A662 camera. 10× objective was used for the photographs seen in FIGS. 21 and 22.

Microscope picture Compound (I) Na.H2O (FIG. 22) shows a needle-like shape morphology, which may not be ideal in pharmaceutical development in relation to tableting performance.

Microscope picture of Compound (I) form A (FIG. 21) displays a crystalline material in a non-needle shape morphology.

### Example 14—MAGL Data for Compound (I)

### Biological Evaluation

Compound (I) was tested to assess its MAGL and serine hydrolase inhibitory activity using the following in vitro and in vivo assays.

### In Vitro Competitive Activity-Based Protein Profiling

Proteomes (mouse brain membrane fraction or cell lysates for mouse assays; human prefrontal cortex or cell membrane fractions for human assays) (50 μL, 1.0 mg/mL total protein concentration) were preincubated with varying concentrations of inhibitors at 37° C. After 30 min, FP-Rh or HT-01 (1.0 μL, 50 μM in DMSO) was added and the mixture was incubated for another 30 min at 37° C. Reactions were quenched with SDS loading buffer (15 μL-4×) and run on SDS-PAGE. Following gel imaging, serine hydrolase activity was determined by measuring fluorescent intensity of gel bands corresponding to MAGL using ImageJ 1.43u software.

Preparation of Mouse Brain Proteomes from Inhibitor Treated Mice

Inhibitors were administered to wild-type C57Bl/6J by oral gavage in a vehicle of polyethylene glycol. Each animal was sacrificed 4 h following administration and brain proteomes were prepared and analyzed according to previously established methods (See Niphakis et al., ACS Chem. Neurosci. 2011 and Long et al. Nat. Chem. Biol. 2009).

Compound (I) demonstrated activity in the assays described herein as indicated in Table 2.

TABLE 2
MAGL inhibition (%) and IC₅₀ data for Compound (I)

| | MAGL % | MAGL | MAGL % |
|---|---|---|---|
| | inh. 1 μM | IC₅₀ | inh. 5 mg/kg |
| Example | (human) | (human) | (mouse) |
| Compound | A | **** | A |
| (I) | | | |

****IC₅₀ is less than or equal to 50 nM;
A = % inhibition is greater than or equal to 75%.

1. (canceled)

2. A crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form A having a crystal form characterized by an XRPD obtained using CuK_{α1} radiation (λ=1.5406 Å) showing peaks at the following 2θ-angles: 10.36, 16.45, 16.71, 19.66, 21.85, and 24.93°.

3. A crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form C having a crystal form characterized by an XRPD obtained using CuK_{α1} radiation (λ=1.5406 Å) showing peaks at the following 2θ-angles: 5.75, 11.08, 12.50, 15.21, 17.58, and 20.06°.

4. A crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form D having a crystal form characterized by an XRPD obtained using CuK_{α1} radiation (λ=1.5406 Å) showing peaks at the following 2θ-angles: 6.12, 12.26, 12.44, 13.42, 18.46, and 19.26°.

5. A crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid form E having a crystal form characterized by an XRPD obtained using CuKα1 radiation (λ=1.5406 Å) showing peaks at the following 2θ-angles: 10.25, 15.41, 16.29, 16.55, 19.56, and 24.72°.

6. A crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid hydrate having a crystal form characterized by an XRPD obtained using CuK_{α1} radiation (λ=1.5406 Å) showing peaks at the following 2θ-angles: 8.84, 14.74, 15.95, 17.42, 20.70, and 22.71°.

7. A crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, wherein the crystalline form is 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid sodium salt monohydrate form having a crystal form characterized by an XRPD obtained using CuK_{α1} radiation (λ=1.5406 Å) showing peaks at the following 2θ-angles: 7.13, 9.03, 12.16, 18.09, 18.47, and 18.89°.

8. A pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt, according to claim 2 and one or more pharmaceutically acceptable carriers or diluents.

9.-15. (canceled)

16. A pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt, according to claim 3 and one or more pharmaceutically acceptable carriers or diluents.

17. A pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt, according to claim 4 and one or more pharmaceutically acceptable carriers or diluents.

18. A pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt, according to claim 5 and one or more pharmaceutically acceptable carriers or diluents.

19. A pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt, according to claim 6 and one or more pharmaceutically acceptable carriers or diluents.

20. A pharmaceutical composition comprising a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid, or a pharmaceutically acceptable salt, according to claim 7 and one or more pharmaceutically acceptable carriers or diluents.

21. A method for the treatment of a disease or a disorder in a patient in need thereof, comprising administering to the patient a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid according to claim 2, wherein the disease or disorder is selected from pain, central pain, epilepsy/seizure disorder, acute repetitive seizures, temporal lobe epilepsy, Dravet syndrome, Lennox Gastaut syndrome or Angelman syndrome, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

22. A method for the treatment of a disease or a disorder in a patient in need thereof, comprising administering to the patient a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid according to claim 3, wherein the disease or disorder is selected from pain, central pain, epilepsy/seizure disorder, acute repetitive seizures, temporal lobe epilepsy, Dravet syndrome, Lennox Gastaut syndrome or Angelman syndrome, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

23. A method for the treatment of a disease or a disorder in a patient in need thereof, comprising administering to the patient a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid according to claim 4, wherein the disease or disorder is selected from pain, central pain, epilepsy/seizure disorder, acute repetitive seizures, temporal lobe epilepsy, Dravet syndrome, Lennox Gastaut syndrome or Angelman syndrome, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

24. A method for the treatment of a disease or a disorder in a patient in need thereof, comprising administering to the patient a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid according to claim 5, wherein the disease or disorder is selected from pain, central pain, epilepsy/seizure disorder, acute repetitive seizures, temporal lobe epilepsy, Dravet syndrome, Lennox Gastaut syndrome or Angelman syndrome, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

25. A method for the treatment of a disease or a disorder in a patient in need thereof, comprising administering to the patient a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid according to claim 6, wherein the disease or disorder is selected from pain, central pain, epilepsy/seizure disorder, acute repetitive seizures, temporal lobe epilepsy, Dravet syndrome, Lennox Gastaut syndrome or Angelman syndrome, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.

26. A method for the treatment of a disease or a disorder in a patient in need thereof, comprising administering to the patient a therapeutically effective amount of a crystalline form of 2-(2-((4-(((1,1,1,3,3,3-hexafluoropropan-2-yl)oxy)carbonyl)piperazin-1-yl)methyl)-5-(trifluoromethyl)phenoxy)-2-methylpropanoic acid according to claim 7, wherein the disease or disorder is selected from pain, central pain, epilepsy/seizure disorder, acute repetitive seizures, temporal lobe epilepsy, Dravet syndrome, Lennox Gastaut syndrome or Angelman syndrome, Alzheimer's disease, Huntington's chorea, Huntington's disease, multiple sclerosis, obsessive-compulsive disorder, Parkinson's disease, depression, post-traumatic stress disorder, generalized anxiety disorder, persistent motor tic disorder, persistent vocal tic disorder, and dystonia.
